(19) United States
(12) Patent Application Publication
(10) Pub. No.: US 2009/0029369 A1
(43) Pub. Date: Jan. 29, 2009

(54) GENETIC SELECTION SYSTEM TO IDENTIFY PROTEASES, PROTEASE SUBSTRATES AND PROTEASE INHIBITORS

(75) Inventors: Valerie Cottier, Zurich (CH); Urs Luthi, Basel (CH); Alcide Barberis, Zurich (CH)

Correspondence Address: MARSHALL, GERSTEIN & BORUN LLP, 233 S. WACKER DRIVE, SUITE 6300, SEARS TOWER, CHICAGO, IL, 60606, US

(73) Assignee: ONCALIS AG, Schlieren (CH)

(21) Appl. No.: 12/088,258

(22) PCT Filed: Sep. 27, 2005
(86) PCT No.: PCT/CH2005/000555

§ 371 Date: Aug. 29, 2008

(51) Int. Cl. C12N 9/00; C12N 9/90; C12N 15/61; C12N 15/81; C12N 1/19; C12Q 1/37; C12Q 1/68

(52) U.S. Cl. 435/6; 435/183; 435/233; 536/23.2; 435/320.1; 435/254.2; 435/23

(57) ABSTRACT

The present invention concerns a tester protein for identifying and/or monitoring protease activity in a cellular assay suitable for high throughput screenings by growth selection, wherein the tester polypeptide is a non-regulatory protein carrying a protease cleavage sequence. Upon co-expression of the protease recognizing said cleavage sequence the tester protein is inactivated, which influences the growth and/or survival of the host cells under the chosen conditions. However, in the presence of protease inhibitor the growth phenotype is reversed. The system can be used to identify proteases, protease inhibitors, and protease cleavage sites.

### TECHNICAL FIELD

The present invention relates to a non-regulatory tester protein comprising a protease cleavage site, a nucleic acid encoding said tester protein and a cell expressing said tester protein; the invention also relates to the use of said tester protein in an assay for identifying and monitoring the activity of cellular proteases, for selecting inhibitors of said proteases based on cell proliferation of a suitable tester strain, and for identifying protease cleavage sequences.

### BACKGROUND ART

Proteases are enzymes which catalyse the splitting of interior peptide bonds in a protein. Many proteases are extracellular for the purpose of the degradation of proteins to amino acids. Other proteases are used during protein targeting, in particular secretion, whereby polypeptide precursors are cleaved specifically to yield the mature forms. For example, a membrane-bound protein can be converted to a soluble form or an inactive precursor molecule can be activated by a functional protease. Such proteases can also be found in organellar compartments or are associated with membranes.

Besides the proteasome, which is a proteolytic enzyme complex that degrades cytosolic and nuclear proteins, there are specific cytosolic proteases which specifically process polypeptides. Well known are the caspases that are activated during apoptosis.

Proteases are also essential for the replication cycle of many viruses. Retroviruses, picornaviruses and herpesviruses for example encode proteins that are synthesised as polyprotein precursors and that are later proteolytically processed to mature viral proteins (Tong 2002). Proteases have also been shown to be physiologically important for bacterial pathogens and are thus implicated in infectious diseases.

Since proteases play a critical role in the regulation of many biological processes, failures in their functioning can lead to severe diseases. Therefore, in the last decades, the pharmaceutical industry has recognised the potential of proteases as targets for drug development. Treatments against cancer, inflammatory, respiratory, cardiovascular and neurodegenerative diseases are being developed on the basis of protease inhibition (Lüthi 2002). To cure hypertension a panel of angiotensin-converting enzyme (ACE) inhibitors have been identified by rational drug design and are nowadays widely prescribed (Hilleman 2000). In the same way, as indeed several viruses depend on the proteolysis of primary polypeptide precursors for their replication, viral proteases are prime therapeutic targets for the treatment of viral diseases, as highlighted by the success story of drugs against human immunodeficiency virus (HIV) (Chrusciel and Strohbach 2004; Randolph and DeGoey 2004).

Besides the HIV protease, many other viral proteases are targets for inhibitor screenings. The human cytomegalovirus (CMV), a member of the herpes virus family, is an opportunistic pathogen that can cause severe illness or death of immunocompromised individuals, such as AIDS patients or recipients of organ and bone marrow transplants (Holwerda 1997; Waxman and Darke 2000). Like the other herpes viruses, it encodes a protease that is essential for the production of infectious virus and that functions during the assembly and maturation of the capsid (Welch, Woods et al. 1991; Sheaffer, Newcomb et al. 2000; Gibson; Trang, Kim et al. 2003). The protease itself is released from the 75 kDa precursor protein upon autoproteolytic cleavage at the maturational (M) and release (R) sites (Baum, Bebernitz et al. 1993). M-type cleavage removes the carboxy-terminal tail, whereas cleavage at the R-site releases the proteolytic domain, also called assemblin. The mature protease contains 256 amino acids, and its catalytic site is formed by the unusual triad His-Ser-His as opposed to classical serine proteases that function with the His-Ser-Asp/Glu triad (Chen, Tsuge et al. 1996; Shieh, Kurumbail et al. 1996). Remarkably, dimerisation is a prerequisite for enzymatic activity (Margosiak, Vanderpool et al. 1996) even though the two catalytic sites have been shown to act in an independent manner (Batra 2001). All herpesvirus protease enzymology and inhibition studies to date have been performed with the 28 kDa mature form (Pinko, Margosiak et al. 1995; Bonneau, Grand-Maitre et al. 1997; Hoog, Smith et al. 1997; Khayat, Batra et al. 2003) though the 75 kDa precursor has been demonstrated to be catalytically active as well (Lawler and Snyder 1999; Wittwer, Funckes-Shippy et al. 2002).

Besides herpesvirus proteases, other viral proteases such as Hepatitis C virus NS3 protease and rhinovirus 3C protease, both of which can be expressed as functional enzymes in yeast, are of interest. In addition, human soluble proteases like caspases, cathepsins (involved in different cancers: (Fehrenbacher and Jaattela 2005)), calpains (responsible for endothelial dysfunction and vascular inflammation: (Stalker, Gong et al. 2005)), or dipeptidyl peptidase IV (main cause of diabetes: (McIntosh, Demuth et al. 2005) are targets for protease inhibitor screens.

Successful application of protease inhibitors in human therapy requires defined properties of drugs, such as membrane permeability, stability and lack of toxicity (Barberis 2002). Most high throughput screening (HTS) campaigns are performed with enzymatic in vitro assays, where compounds are tested exclusively with respect to their potential to inhibit proteolytic activity.

Cellular screening systems provide a promising alternative to screen or select directly for compounds with additional features that are essential for their use as drugs in a cellular context. Indeed, compounds are identified as hits at the condition that they not only inhibit proteolytic activity, but are also stable within the cell, capable of penetrating biological membranes, and exert no or only limited toxic effects on the cell.

Cell-based assays have notable advantages over in vitro assays. First, no purification of enzyme is required, avoiding a time consuming and costly process to obtain an active target. Second, target conformation and activity are examined in a cellular context, closer to natural physiological state than in an in vitro assay.

Several cell-based assays have already been used to screen for protease inhibitors. Most of them rely on a reporter protein that allows a gradual read-out paralleling intracellular protease activity levels. Examples of such reporter proteins are GFP (green fluorescence protein; Lindsten, Uhlikova et al. 2001; Belkhiri, Lytvyn et al. 2002) or SEAP (secreted alkaline phosphatase; Lee, Shih et al. 2003; Mao, Lan et al. 2003; Oh, Kim et al. 2003). However, such systems have the disadvantage, that every toxic compound will also decrease the amount of reporter protein or signal in the medium, just by decreasing the number of cells producing it. By consequence, a high number of false positives will be obtained, which have to be further evaluated at costs of time and resources.

The yeast transcription factor Gal4p has been exploited in different detection systems for protease inhibitors due to its two-domain structural property by inserting the protease target site between the two domains.

Protease activity separates the DNA-binding domain from the activation domain, causing stop of transcription of a Gal4p regulated reporter gene, e.g. lacZ. Protease inhibitors prevent cleavage and therefore inactivation of the Gal4p transcription factor, restoring transcriptional expression. Such systems have been developed for protease 3C from coxsackievirus (Dasmahapatra, DiDomenico et al. 1992) and for cytomegalovirus protease (Lawler and Snyder 1999). In a similar way the herpesvirus transcription factor VP16 was used in combination with a lacZ reporter gene to detect CMV protease activity. Other hybrid regulatory protein/reporter gene combinations have been used in various ways (U.S. Pat. No. 5,721,133; US2004042961; U.S. Pat. No. 6,117,639; U.S. Pat. No. 6,699,702).

Recently discovered protease inhibitors are among the more promising antiviral drugs; yet, there is still a need for more and alternative protease inhibitors, and thus for HTS systems enabling the rapid and efficient identification of new antiviral drugs. Whereas primarily mammalian or insect cells have been used in past screening campaigns (Johnston 2002; Kemnitzer, Drewe et al. 2004; Zuck, Murray et al. 2004), yeast cells provide an alternative model with several technical advantages. The fast and inexpensive cultivation, the easy genetic manipulation and the high degree of conservation of basic molecular mechanisms make this eukaryotic organism a valuable tool for drug screening (Botstein, Chervitz et al. 1997; Munder and Hinnen 1999; Brenner 2000; Hughes 2002). In addition, yeast provide a heterologous, yet eukaryotic-environment, suitable for preventing redundant processes and for supplying a null background for the expression of several human targets. Of course, despite the high degree of similarity of basic cellular processes between yeast and human cells, yeast show some differences that might impair attempts to reproduce the activity of some target proteases. However, as long as the appropriate controls are respected, the employment of yeast in cell-based assays has many advantages, in particular for HTS.

Another improvement in the search of antiviral compounds would be to have a selection rather than a screening procedure, wherein only those cells survive that are exposed to an inhibitor. Such a selection system has been developed in yeast by using the Gal4p carrying a tobaccho etch virus (TEV) protease cleavage sequence between its two domains and measuring the lack of Gal4 regulatory function upon cleavage by the TEV protease as the lack of growth on the suicide substrate 2-deoxygalactose (Smith, T. A. and Kohorn, B. D., 1991). This system allows for the positive selection of inhibitors. However, the system has two further disadvantages: (i) it requires the addition of a toxic compound to the medium, and (ii) it uses a transcriptional regulatory protein, which only indirectly, i.e. by control of transcription of other genes leads to the desired phenotype, thus increasing the possibility to identify false positives.

A drug that inhibits a viral protease can be used to prevent production of new infectious viral particles. However, the efficacy of such drugs, when they are prescribed in monotherapy and especially in low dose therapy, is often limited by the rapid emergence of drug resistant strains. In the case of HIV, mutations at several key amino acid residues of the protease, which abolish protease inhibition by already marketed drugs, have been described. The occurrence of drug resistant strains is increasing, and the phenomenon of cross-resistance is gaining importance. Therefore, new drugs against such proteases, with different modes of action, are needed. Currently, most protease inhibitors are complex peptidomimetic compounds with poor aqueous solubility, low bioavailability and short plasma half-lives. The complexity of these agents not only contributes to their high cost but also increases the potential for unwanted drug interactions. There is a need for novel compounds working as protease inhibitors in the context of many widely spread diseases. In order to find such drugs, there is also a need for biological systems, in particular selection rather than screening systems allowing by simple and reliable in vivo tests to select for protease inhibitors in high throughput screenings.

### DISCLOSURE OF THE INVENTION

Hence, it is a general object of the invention to provide a non-regulatory tester polypeptide for monitoring protease activity, which can be used in a protease inhibitor selection system and for the identification of proteases and protease cleavage sequences.

Now, in order to implement these and still further objects of the invention, which will become more readily apparent as the description proceeds, the tester polypeptide is manifested by the features that it
comprises the sequence of a marker protein whose activity can be detected by positive and/or negative growth selection and an additional sequence, whereby said additional sequence is inserted at a specific permissible site in a surface loop of said marker protein and comprises a cognate cleavage sequence for a protease, and
is inactivated upon cleavage by said protease.

The tester polypeptide of the present invention comprises a marker protein with a detectable activity, modified by an insertion of a cleavage sequence for a protease which creates an in frame fusion polypeptide that is still functional. Upon cleavage of the polypeptide by the matching protease the tester polypeptide is inactivated. The tester polypeptide as well as the marker protein of the present invention are non-regulatory, i.e. are not transcriptional regulators of gene expression.

The marker protein of the present invention can either have a metabolic enzymatic activity or can be a structural protein. If inactivation of said marker protein causes a deficiency of cellular growth, this allows a positive selection for the presence of said marker protein. This effect can depend on the growth conditions. The marker protein can also be a negative selection marker that has an activity leading to growth inhibition. For example, it can be an enzymatic activity catalyzing the conversion of a non-toxic substrate into a toxic product. Cells comprising said activity die, whereas cells lacking said activity survive.

In a preferred embodiment of the present invention the marker protein is a cytoplasmic protein. Preferably, it is an enzyme of a biosynthetic pathway for an essential cellular compound, for example an amino acid, nucleotide, lipid or cofactor. More preferably, it is an enzyme of an amino acid biosynthesis pathway, such as the tryptophan biosynthesis pathway. Most preferably, the essential protein is the Trp1p of yeast, which catalyses the isomerisation of N-(5′-phosphoribosylanthranilate in the biosynthesis of the amino acid tryptophan that is essential for cell proliferation. Under tryptophan deficient growth conditions cells therefore can survive only if all of their enzymes involved in tryptophan biosynthesis, including Trp1p or the Trp1p derived tester protein, are functionally active.

In another preferred embodiment of the present invention the marker can be used for both positive and negative selection. This is possible, for example, for the preferred marker of the present invention, the Trp1p protein encoded by the TRP1 gene. This enzyme is required for the conversion of anthranilic acid to tryptophan, and thus is a typical auxotrophy marker allowing positive selection. The antimetabolite 5-fluoroanthranic acid (FAA) was found to be particularly effective for TRP1 counter selection, as it is converted in the presence of Trp1p to the toxic 5-fluorotryptophan. Therefore, the Trp1p marker can also be used for negative selection (Toyn et al. 2000).

Alternatively, the yeast URA3 gene product orotidine 5′ decarboxylase required for uracil biosynthesis can be used for positive as well as for negative selection. In a positive selection, cells are grown on media lacking uracil, which allows growth of only those cells that express a functional enzyme. A negative selection can be performed on media containing 5-fluoroorotic acid (5-FOA), because the URA3 gene product converts 5-FOA to a toxic compound. Therefore, cells expressing a functional enzyme cannot grow.

Another example is the yeast Gal1p protein galactokinase, which converts galactose to galactose-1-phosphate. This intermediate is converted by the GAL7 encoded transferase into glucose-1-phosphate, which is metabolized. The Gal1p protein is thus essential for growth of yeast with galactose as the only carbon source, allowing positive selection. In addition, in yeast cells lacking the transferase enzyme encoded by the GAL7 gene expression of GAL1 leads to accumulation of the intermediate galactose-1-phosphate to toxic levels, thus allowing a negative selection (Gunde et al. 2004).

Another preferred marker for negative selection is the CYH2 gene, encoding the ribosomal protein Rpl28. Yeast cells carrying a mutation in their endogenous CYH2 allele are resistant to the antibiotic cycloheximide, whereas cells expressing wild-type CYH2 are sensitive.

In another preferred embodiment of the pre-sent invention the protease cleavage sequence has a size of 5-39 amino acids. For inactivation of the tester polypeptide, the protease cleavage sequence and the corresponding protease recognising and cleaving said sequence must be present in the system together in the same cellular compartment. It is possible that a protease requires a minimal cleavage sequence of only a few amino acids, or even only a single amino acid, like for example the dipeptidyl peptidase IV, which is a post-proline cleaving enzyme. However, it is also possible that in the context of another polypeptide, into which a protease cleavage sequence is inserted, a longer extension of said cleavage sequence can be cleaved more efficiently. In some cases the minimal cleavage sequence may not be known and therefore just any number of amino acids encompassing the cleavage site within a natural target polypeptide of a specific protease may be chosen.

In a more preferred embodiment the protease cleavage site has a sequence selected from the group of cleavage sequences listed in Table 1. Most preferred are the cleavage sequences SEQ. ID. NO:1=GGVVNASCRLAGG, its longer version SEQ. ID. NO:2 PTALLSGGAKVAERAQAGVVNASCRLATASGSEAATAGP, SEQ. ID. NO:3=KVAERANAGVVQASCRLATAS, which are all recognised by human cytomegalus virus (CMV) protease. Table 1 summarises a number of known proteases and their cognate target cleavage sequences that are in the scope of the present invention; however, these pairs are only examples and in no way exclusive or anyhow limiting.

TABLE 1
Proteases and their cognate cleavage sequences

| | | |
|---|---|---|
| | Cleavage sequence and | SEQ. ID. |
| Proteases | site of cleavage (↓) | NO: |

Herpes virus
proteases:

| | | |
|---|---|---|
| CMV (human | GVVNA↓SCRLA | 1 |
| cytomega- | GGVVNA↓SCRLAGG | 2 |
| lovirus | KVAERANAGVVQA↓SCRLATAS | 3 |
| | PTALLSGGAKVAERAQAGVVNA↓S | 4 |
| | CRLATASGSEAATAGP | |
| | VXA↓S; LXA↓S; IXA↓S | 5; 6; 7 |
| HSV1 (herpes | ALVNA↓SSAAHV | 8 |
| simplex virus | | |
| type 1) | | |
| VZV (varicella | QDVNA↓VEASS | 9 |
| zoster virus) | | |
| EBV (Epstein- | KLVQA↓SASGVA | 10 |
| Barr virus) | | |
| HHV-6 (human | PSILNA↓S | 11 |
| herpes virus | | |
| 6) | | |
| Other virus | | |
| proteases: | | |
| HIV-1 | SFNF↓PQIT; TLNF↓PISP | 12; 13 |
| Hepatitis C | DLEVVT↓STWVL | 14 |
| (NS3/4A) | | |
| Coxsackievirus | GTTLEALFQ↓GPPV | 15 |
| 3C | | |
| Rhinovirus 3C | LEVLFQ↓GPLG | 16 |
| SARS corona- | SAVLQ↓SGF | 17 |
| virus 3C-like | | |
| proteinase | | |
| Caspases: | | |
| Caspase-1 | WFKD↓S; FEDD↓A; YVHD↓A; | 18; 19; 20 |
| (ICE) | DGPD↓G; DEVD↓G | 21; 22 |
| Caspase-2 | DEVD↓G | 22 |
| Caspase-3 | IETD↓S; DGPD↓G; DEVD↓G | 23; 21; 22 |
| | DEVD↓N; DMQD↓N; DEPD↓S | 24; 25; 26 |
| | DEAD↓G; DETD↓S; DACD↓T | 27; 28; 29 |
| Caspase-6 | DGPD↓G; DEVD↓G; VEID↓N | 21; 22; 30; |
| Caspase-7 | DEVD↓G | 22 |
| Caspase-8 | VETD↓S; LEMD↓L | 31; 32 |
| (FLICE) | | |
| Caspase-9 | DEVD↓G | 22 |
| Other | | |
| proteases: | | |
| Plasmepsin | ERMF↓LSFP | 33 |
| Thrombin | VPR↓SFR | 34 |
| ACE (Angio- | RPPGFSP↓FR | 35 |
| tensin I- | | |
| converting | | |
| enzyme) | | |
| Cathepsin S | | |
| Cathepsin K | | |
| MMP2 | | |
| MMP7 | GPLG↓VRGL | 36 |
| MMP13 | | |
| Bacillus | LARRKPVLP↓ALTINP | 37 |
| anthracis | | |
| lethal factor | | |
| Renin | PFHL↓LVYS | 38 |
| Dipeptidyl | | |
| peptidase IV | | |

In another preferred embodiment of the pre-sent invention the protease cleavage sequence is inserted into a surface loop of the essential protein such that it does not interfere with the function of the protein, as it does not significantly affect the folding of the essential protein. The candidate surface loops of an essential protein can either be known if the structure of said protein is known, or they can be predicted if the structure of a related protein is known. In addition, they can be predicted from computer generated secondary structure predictions and hydrophobicity analysis based on the polypeptide sequence. Often ideal insertion sites are at glycine or proline residues in sequence stretches that connect alpha helices and/or beta sheets and that are hydrophilic. Once a cleavage sequence of a known protease is inserted into a putative permissible surface loop of an essential protein, the activity of the resulting tester polypeptide is compared to the activity of the corresponding unmodified essential protein by measuring cell proliferation. A permissible site of the fusion will allow cell growth under the relevant conditions when the tester polypeptide is expressed, whereas a non-permissible site will lead to lack of cell growth. In a final step of validation it has to be tested whether the protease is able to recognise and cleave the fusion protein comprising the cleavage site. Hence, in the presence of the corresponding protease the protein should be cleaved inside the cell, which can be investigated for example by Western blot analysis or by cell growth selection. This is the case for the example of the yeast Trp1p, which tolerates the insertion of a protease cleavage sequence after amino acid Gly194, said sequence being recognised and cleaved by its cognate protease, thus leading to cell death. Hence, the use of the insertion site after Gly194 of the yeast Trp1p protein for insertion of a protease cleavage sequence is a preferred embodiment of the present invention.

It is also comprised by the present invention that single or multiple point mutations within the essential protein and/or within the protease cleavage sequence of the present invention are used to improve the system. For example, the insertion of a cleavage sequence may have some impact on the folding and/or activity of the essential protein, which might be compensated by additional mutation(s). Any mutations can be introduced as long as the function of the tester protein in cell proliferation and the susceptibility of the cleavage sequence to the protease are not disturbed. Therefore, one or more point mutations, which can also be insertion or deletion mutations, fulfilling these requirements are envisaged in a further preferred embodiment of the present invention. Most preferred are one or more point mutations in the form of altered amino acids within the natural cognate cleavage sequence of a given protease.

In another preferred embodiment the inserted sequence is the target sequence of a viral protease. Most preferably said viral protease is the human CMV protease.

In another preferred embodiment of the pre-sent invention the inserted sequence encodes an autoprotease and comprises the cleavage sequence for said autoprotease. An autoprotease is a protein that cleaves at least one site of its own sequence in a self-processing manner. Many viral precursor proteins comprise autoprotease activities that lead to processed products of the precursor molecule. The preferred autoprotease of the present invention is the autoprotease 3C from coxsackievirus.

Also a subject of the present invention is a nucleic acid encoding the tester polypeptide of the pre-sent invention. Preferably, said nucleic acid is a DNA. Said DNA comprises the gene with or without a promoter for expression of said tester polypeptide.

In a preferred embodiment of the present invention said DNA is part of a recombinant vector comprising transcriptional start and termination signals in order to allow expression of said tester protein. If said promoter is a regulated promoter, it is possible to optimise expression of said tester protein in order to optimise the ratio of tester protein to protease. Regulated promoters are well known to the person skilled in the art. The use of a regulated promoter depends on the cellular system in which the tester polypeptide is expressed. For example, if a bacterial cell is used, a lac or tac promoter may be used that is inducible by addition of isopropyl-β-D-thiogalactopyranoside (IPTG), or the ara promoter that is induced by the addition of arabinose and repressed by the addition of glucose. If a yeast cell is used, a suitable regulated promoter may be the galactose inducible GAL1 promoter, the copper inducible CUP1 promoter, the PHO5 promoter inducible by phosphate starvation, the HSP70 (heat shock) promoter inducible by increase of temperature, MET promoters inducible by methionine, or the CYC1 promoter that is induced by oxygen and repressed by glucose. This list of promoters is by far not complete and many other known promoters can be used as well within the scope of the present invention.

It is also possible that the DNA of the pre-sent invention is integrated into the host chromosome. In this case, the promoter must be comprised by said DNA, or it must be provided by the host DNA flanking the site of said integration.

The present invention also provides a prokaryotic or eukaryotic cell comprising the nucleic acid of the present invention and a protease. Said nucleic acid is transformed into said cell and either propagated as an extra-chromosomal element, or integrated into the chromosome of said cell. Expression of a protease in said cell is driven by a promoter that can be constitutive or regulated. In a preferred embodiment of the present invention an inducible promoter will be used, which allows to control the amount of synthesised protease for adaptation to the amount of tester polypeptide produced by said cell. The protease is encoded on an expression plasmid that is transformed into said cell. Alternatively, a protease naturally expressed in said cell is used.

The cloning of genes coding for a tester protein or a protease is done by gene synthesis and routine techniques including PCR known to the skilled person using known sequences of said proteins.

A further aspect of the present invention is the identification of a protease inhibitor by a method comprising the steps of
providing a cell of the present invention comprising a tester protein with a protease cleavage sequence and comprising a matching protease,
exposing said cell to candidate inhibitor substances,
growing said cell under conditions that are non-permissive for cell proliferation in the presence of a functional protease, but permissive for cell proliferation in the additional presence of an inhibitor of said protease, and
selecting an inhibitor on the basis of cell proliferation.

Candidate inhibitor molecules can be members of known chemical compound libraries, molecules from a random peptide library or natural products isolated from microorganisms, fungi, plants or animals, from water, soil or any natural environment where these organisms live. Preferably, these molecules are able to penetrate the cell wall and reach the cytosol, where they can block the protease or mask the protease cleavage site on the tester protein. Alternatively, derivatives of known protease inhibitor molecules can be tested. Preferably, the method is based on yeast cells. More preferably a yeast mutant deficient in the multi drug export systems encoded by the genes pdr5, snq2, and yor1 is used as a host.

In a preferred embodiment of the present invention, cells are exposed to putative inhibitory molecules before or at the time when they are shifted to conditions that are non-permissive for cell proliferation in the presence of a functional protease. This will eliminate candidate inhibitors which are per se toxic for the cell, i.e. which block other essential cellular functions. In another preferred embodiment of the present invention the protease is provided by expressing it under the control of a regulated promoter, for example the yeast Gal1 promoter. This allows to chose expression levels of the protease in accordance with the concentration of inhibitor. For example, low levels of protease expression can be used when weak inhibitors are preferred, whereas high levels of protease are useful to detect strong inhibitors. Moreover, this also allows to choose inhibitor concentrations in an non-toxic range.

The inhibitor selection system of the present invention comprises the possibility to manipulate the levels of tester protein as well as the levels of protease and can therefore be optimised in various ways. A further aspect of the present invention is the use of the inhibitor selection system in high throughput (HT) assays. The output signal of the assay, i.e. the turbidity of the cell culture can be measured directly in a single step in the microtiter plate by measurement of light absorption or light scattering without the use of special equipment or the need for additional chemicals and/or additional handling.

Another aspect of the present invention is to provide a method to identify a suitable site in a non-regulatory marker protein for insertion of a protease cleavage sequence, said marker protein being suitable for positive as well as negative growth selection. In said method the protease is modulated on the one side at the level of its presence or absence or at the level of its expression or at the level of its activity, and on the other side a positive as well as a negative selection step are used in a successive given order. This leads to several alternative embodiments of the present invention:

a) if an inhibitor of said protease is available, the method comprises the steps of
identifying putative surface loops in the marker protein,
providing an expression vector comprising a nucleic acid encoding said marker protein,
inserting a nucleic acid comprising a coding sequence of said protease cleavage sequence at a random position within the coding sequence of said putative surface loops, resulting in a plasmid comprising a gene encoding a candidate tester protein as defined above,
transforming with said plasmid a yeast cell comprising a protease that is capable of cleaving said protease cleavage sequence,
growing transformants in the presence of a specific inhibitor of said protease under conditions requiring a function of said tester protein (positive selection),
shifting growing clones to conditions non-permissive for a function of said tester protein and lacking an inhibitor (negative selection),
determining the nucleic acid sequence of the gene encoding said tester protein of a surviving clone.

Transformants are cells that have stably taken up DNA during transformation. If not otherwise mentioned, plasmids used for transformations in the scope of the present invention carry a selectable marker, and transformants can be obtained under corresponding selective conditions.
b) In the absence of a known inhibitor of said protease, the identification of a suitable insertion site in a non-regulatory marker protein as defined above can be achieved by a method comprising the steps of
identifying putative surface loops in said marker protein,
providing an expression vector comprising a nucleic acid encoding said marker protein,
inserting a nucleic acid comprising a coding sequence for said protease cleavage sequence at a random position within the coding sequence of said putative surface loops, resulting in a plasmid comprising a gene encoding a candidate tester protein,
transforming with said plasmid a yeast cell comprising a gene encoding a protease that is capable of cleaving said protease cleavage sequence, said gene being under the control of a tightly regulated promoter,
growing transformants under repressing or non-inducing conditions with respect to said promoter and under conditions requiring the function of the tester protein (positive selection),
shifting growing cells to derepressing or inducing conditions with respect to said promoter for protease expression and to non-permissive conditions with respect to a function of said tester protein (negative selection),
determining the nucleic acid sequence of the gene encoding said tester protein of a growing cell.

In an alternative embodiment of the present invention, instead of a single cell with an inducible promoter for protease expression two cells are used for the selection, and in this case the method comprises either the steps of c)
identifying putative surface loops in said marker protein,
providing an expression vector comprising a nucleic acid encoding said marker protein,
inserting a nucleic acid comprising a coding sequence for said protease cleavage sequence at a random position within the coding sequence of anyone of said putative surface loops, resulting in a plasmid comprising a gene encoding a candidate tester protein,
providing a first yeast cell comprising a protease capable of cleaving said cleavage sequence and a second yeast cell lacking said protease,
transforming said first yeast cell with said plasmid and growing transformants under non-permissive conditions with respect to a function of said tester protein (negative selection),
isolating said plasmid from a surviving cell,
transforming said second yeast cell with said isolated plasmid and growing transformants under conditions requiring a function of said tester protein (positive selection),
determining the nucleic acid sequence of said gene encoding said tester protein of a growing cell,

or it comprises the steps of d)
identifying putative surface loops in said marker protein,
providing an expression vector comprising a nucleic acid encoding said marker protein,
inserting a nucleic acid comprising a coding sequence for said protease cleavage sequence
at a random position within the coding sequence of anyone of said putative surface loops, resulting in a plasmid comprising a gene encoding a candidate tester protein,

providing a first yeast cell comprising a protease capable of cleaving said cleavage sequence and a second yeast cell lacking said protease,
transforming said second yeast cell with said plasmid and growing transformants under conditions requiring a function of said tester protein (positive selection),
isolating said plasmid from a growing cell,
transforming said first cell with said isolated plasmid and growing transformants under conditions non-permissive for a function of said tester protein (negative selection),
determining the nucleic acid sequence of said gene encoding said tester protein of a surviving cell.

Yet another alternative is the use of a single cell lacking said protease and applying a positive selection followed by the introduction of an expression plasmid encoding said protease into the growing cell and then applying a negative selection, i.e. a method comprising the steps of e)
identifying putative surface loops in said marker protein,
providing an expression vector comprising a nucleic acid encoding said marker protein,
inserting a nucleic acid comprising a coding sequence for said protease cleavage sequence at a random position within the coding sequence of anyone of said putative surface loops, resulting in a plasmid comprising a gene encoding a candidate tester protein,
providing a yeast cell lacking a protease capable of cleaving said cleavage sequence,
transforming said yeast cell with said plasmid and selecting for growth under conditions requiring a function of said tester protein (positive selection), obtaining transformants,
providing a second plasmid capable of expressing a gene encoding said protease, transforming said transformants with said second plasmid and selecting for growth under conditions non-permissive for a function of said tester protein (negative selection),
determining the nucleic acid sequence of said gene encoding said tester protein of a surviving cell.

In a preferred embodiment of the present invention the marker protein is a single domain protein. However, multi domain proteins may also be used. In this case, a suitable surface loop can also be within the sequence connecting two domains.

In a similar way, if an inhibitor of the protease is known, it is also possible according to the present invention to identify the cleavage sequence of a known protease by a method comprising the steps of a)
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid comprising a gene encoding a tester protein,
transforming with said plasmid a suitable host cell comprising said protease,
growing transformants in the presence of a specific inhibitor of said protease under conditions requiring a function of said tester protein,
shifting growing clones to conditions non-permissive for a function of said tester protein and lacking said inhibitor,
determining the nucleic acid sequence of the gene encoding said tester protein of a surviving clone.

However, in the absence of an inhibitor of said protease the cleavage site of said protease can be determined by one of the following four variations of the method, namely a method comprising the steps of b)
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid comprising a gene encoding a tester protein,
transforming with said plasmid a suitable host cell comprising the gene encoding said protease under a control of a tightly regulated promoter,
growing transformants under repressing or non-inducing conditions with respect to said promoter and under conditions requiring a function of said tester protein (positive selection),
shifting growing cells to derepressing or inducing conditions with respect to said promoter and non-permissive conditions with respect to a function of said tester protein (negative selection),
determining the nucleic acid sequence of the gene encoding said tester protein of a surviving cell,

or a method comprising the steps of c)
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid comprising a gene encoding a tester protein,
providing a first yeast cell comprising a protease capable of cleaving said cleavage sequence and a second yeast cell lacking said protease,
transforming said first yeast cell with said plasmid and growing transformants under non-permissive conditions with respect to a function of said tester protein (negative selection),
isolating said plasmid from a surviving cell,
transforming said second cell with said isolated plasmid and growing transformants under conditions requiring a function of said tester protein (positive selection),
determining the nucleic acid sequence of the gene encoding said tester protein of a growing cell,

or a method comprising the steps of d)
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid comprising a gene encoding a tester protein,
providing a first yeast cell comprising a protease capable of cleaving said cleavage sequence and a second yeast cell lacking said protease,
transforming said second yeast cell with said plasmid and growing transformants under conditions requiring a function of said tester protein (positive selection),
isolating said plasmid from a growing cell,
transforming said first yeast cell with said isolated plasmid and growing transformants under non-permissive conditions with respect to a function of said tester protein (negative selection),
determining the nucleic acid sequence of said gene encoding said tester protein of a surviving cell,

or a method comprising the steps of e)
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid comprising a gene encoding a tester protein,
providing a yeast cell lacking a protease capable of cleaving said cleavage sequence,
transforming said yeast cell with said plasmid and selecting for growth under conditions requiring a function of said tester protein (positive selection), obtaining transformants,
providing a second plasmid capable of expressing a gene encoding said protease,
transforming said transformants with said second plasmid and selecting for growth under conditions non-permissive with respect to a function of said tester protein.

(negative selection),
determining the nucleic acid sequence of said gene encoding said tester protein of a surviving cell.

A variation of the method to determine the cleavage site of a protease is possible if the non-regulatory marker protein is only used for positive selection. In this case, after the first step of positive selection for cells expressing a functional tester polypeptide the transformants are picked and each split into two identical cell populations, of which one is trans-formed subsequently with the second plasmid expressing a gene encoding said protease, and the other one is trans-formed with the empty vector, i.e. the vector not comprising the gene encoding said protease. The growth of the two transformed populations is then compared under positive selection conditions, and those clones are of interest which do not grow in the presence, but do grow in the absence of said protease. However, this method is preferably only used if few clones are investigated, as it involves more handling than the method using the negative selection. This may be the case if there is already some preliminary information on the protease cleavage sequence but better knowledge is desired. For example, the validation of specific point mutations in a known cleavage sequence may be done with the positive selection method.

Another aspect of the present invention is to provide a method to identify new proteases for a known protease cleavage sequence, said method comprising the steps of
providing cells expressing a functional, non-regulatory tester polypeptide suitable for negative selection,
providing an expression library comprising putative genes encoding said protease,
transforming said cells with said expression library,
growing transformants under non-permissive conditions with respect to a function of said tester protein (negative selection),
identifying among surviving clones those which lack full-length tester polypeptide,
determining from identified clones the nucleic acid sequence of the gene encoding said protease.

Preferably, said expression library expresses proteins from the same organism and/or tissue from which the cleavage sequence has been obtained. Most preferred is a human cDNA library.

As the present invention provides a system comprising a tester protein with a protease cleavage sequence on the one hand and a protease on the other hand as outlined above, this system can be further adapted to specific uses such as the engineering of improved proteases or changing the specificity of a protease. For example, a protease A with specificity for a cleavage sequence B can be co-expressed in a cell with a tester protein comprising a protease cleavage sequence C according to the present invention, and the gene encoding the protease A can be subjected to random or site specific mutagenesis to select for clones that change the protease A such that it can recognize and cleave the cleavage site C. This is possible because the system of the present invention is based on selection, in particular if the tester protein is a genetic marker that allows positive as well as negative selection.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1A depicts a structural model of N-(5′ phosphoribosyl)-anthranilate (Trp1p), a yeast protein essential for cell proliferation, showing the predicted alpha helices, beta sheets and the intervening surface loop regions. FIG. 1B shows a Kyte Doolittle hydropathy plot wherein the tested sites of insertions are indicated.

FIG. 2 shows a spotting assay (FIG. 2A) for evaluating the functionality of Trp1p tester proteins comprising an inserted protease cleavage sequence and the quantified results (FIG. 2B).

FIG. 3 shows the quality of human CMV protease cleavage of a Trp1p tester protein. In FIG. 3A the inserted cleavage sequences a)=wild-type 13-mer (TRP1(194)-M (short); Seq. Id. No:2), b)=wild-type 39-mer (TRP1(194)-M (long); Seq. Id. No:4) and c) mutant 39-mer (TRP1(194)-M (Ala→Glu) (long); Seq. Id. No. 39) are shown. FIG. 3B shows the quantification of cleavage in an experiment using human CMV (HCMV) protease and Trp1p tester polypeptides comprising the different cleavage sequences. FIG. 3C shows biochemical evidence for cleavage of the substrate by human CMV protease in a Western blot experiment following the disappearance of the full-length substrate TRP1(194)-M.

FIG. 4 shows the gradual, reciprocal correlation between human CMV protease expression level and cell growth measured as a result of the protease assay.

FIG. 5 illustrates the validation of the TRP1(194)-M system with known cellular protease inhibitors. FIG. 5A shows the application of the protease inhibitors BI31 and BI36 in the CMV protease inhibitor selection system. FIG. 5B shows the inhibition of cleavage of the Trp1(194)-M (long) substrate by CMV protease in a Western blot.

FIG. 6 shows the growth inhibition of TRP1(194)-2C/3A transformed RLY07 cells by coxsackievirus 3C protease that inactivates the Trp1p tester protein substrate. A comparison of active versus inactive CVB3 3C protease is shown.

### MODES FOR CARRYING OUT THE INVENTION

In the following a cell-based system is described, which enables monitoring protease activity and, in addition, selecting for inhibitors of given proteases.

In this assay, the protease cleavage sequence of interest is inserted into a protein essential for proliferation of yeast cells, the Trp1p protein, yielding the tester protein. Co-expression of the protease with this engineered substrate reduces cell proliferation in selective medium, as it will be shown with the human cytomegalovirus (CMV) protease. In a proof-of-principle experiment, it was demonstrated that a small molecule CMV protease inhibitor prevents inactivation of the modified Trp1p tester protein by blocking of said protease, thus stimulating cell proliferation.

Growth markers impose themselves as the best candidates for the choice of the essential protein for this system. Indeed, most laboratory strains are already deleted for growth markers, allowing for the application of such a system in almost any genetic background. Among these growth markers the N-(5′-phosphoribosyl)anthranilate isomerase (Trp1p) enzyme has been intensively studied (Eder and Kirschner 1992; Eder and Wilmanns 1992; Hommel, Eberhard et al. 1995; Hennig, Sterner et al. 1997), and its 3-dimensional structure from different organisms has been determined. Trp1p is a small monomeric protein that catalyses the isomerisation of N-(5′-phosphoribosyl)-anthranilate in the biosynthesis of tryptophan, an essential amino acid for cell proliferation. Therefore, Trp1p is essential for proliferation of yeast cells when tryptophan is not provided externally. Trp1p was chosen as the essential protein, and was now modified to become the tester protein of choice such that it comprises a protease recognition and cleavage sequence at a permissible site, yet retains its function.

1. Material and Methods

1.1. Yeast Strains

The three major ABC transporter proteins Pdr5p, Snq2p and Yor1p were deleted in the S. cerevisiae JPY5 strain (MATα ura3-52 his3Δ200 leu2Δ1 trp1Δ63 lys2Δ385) to generate the RLY07 strain (MATα ura3-52 his3Δ200 leu2Δ1 trp1Δ63 lys2Δ385 pdr5Δ snq2Δ and yor1Δ) Fusion proteins used in this study were expressed in RLY07.

1.2. Recombinant Plasmids

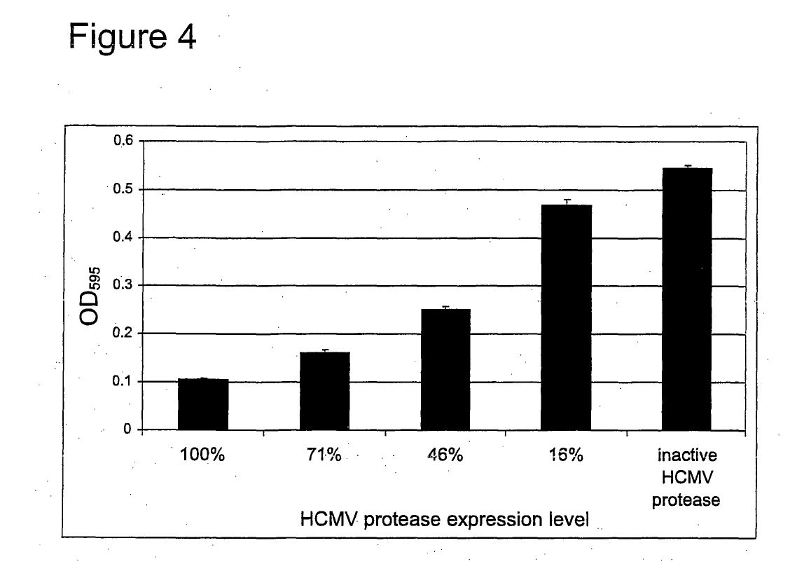

All TRP1-M constructs used in this study were subcloned in the CEN4-ARS1 plasmid pMH4 that contains a LEU2 auxotrophic marker and a polylinker with unique XbaI and SalI restriction sites. Expression of the subcloned TRP1-M constructs is under control of the ADH1 promoter and the GAL11 terminator. The TRP1 gene was amplified by PCR from the YCplac22 plasmid (Gietz and Sugino 1988). The human CMV protease cleavage sequence GGVVNA↓SCRLAGG (derived from the M-site), flanked by NcoI at the 5′-end and NotI at the 3′-end, is inserted after amino acids 49, 102, 132, 165 and 194 of Trp1p. The longer human CMV cleavage sequence (39 amino acids surrounding the M-site) was obtained by PCR amplification of the UL80 gene and subcloned in the previously described TRP1¹⁹⁴-M plasmid via NcoI and NotI restriction sites. The 3C cleavage sequence of coxsackievirus B3 (GTTLEALFQ↓GPPV), which is located at the junction of the viral proteins 2C and 3A was subcloned in TRP1 after amino acid Gly194. HA tags have been added at both the N- and C-terminus of the TRP1(194)-M construct for Western blot analysis. The CMV protease gene encoding amino acids 1-256 of the 75 kDa precursor was obtained by PCR from CMV infected MRC5 human cells and subcloned via unique XbaI and NotI restriction sites in pMH51. pMH51 is an CEN4-ARS1 plasmid that contains a URA3 marker and a full-length GAL1 promoter (100%). For the experiment described in FIG. 4, the CMV protease gene was subcloned on a plasmid series, which contain modified GAL1 promoters that express the protease with 71%, 46% and 16% protein production relative to the original full-length (100%) GAL1 promoter. To subclone the 3C protease gene from the coxsackievirus strain B3, RNA was isolated from an infected HeLa cell culture with FastRNA® Kit-Red from BIO 101. A reverse transcription reaction was performed and 3C encoding DNA fragment was amplified by PCR and cloned via XbaI and NotI sites in a CEN4-ARS1 plasmid that carries a URA3 marker and controls expression by the GAL1 promoter and the GAL11 terminator.

Clonings were done using standard molecular biology techniques (Sambrook & Russell, 3^{rd} ed. 2001, Molecular Cloning, A Laboratory Manual).

1.3. Yeast Media and Transformation

All media were prepared according to Burke et al. (Burke, Dawson et al. 2000). Transformation of yeast cells was performed following the lithium acetate method (Gietz, St Jean et al. 1992).

1.4. Spotting Assay

RLY07 cells transformed with the different TRP1-M constructs were inoculated in 3 ml of 2% -leu glucose medium and grown overnight at 30° C. to saturation. Next morning, cells were diluted in the same medium to OD600 0.25 and grown to OD₆₀₀ 1. Cultures were then washed with 5 ml H2O, resuspended in 2% -leu -trp glucose medium and diluted to 10⁶ cells/ml. 10 μl of serially diluted cultures were spotted on non-selective (-leu) and selective (-leu -trp) 2% glucose plates and incubated during 3 days at 30° C.

1.5. Liquid Growth Assays

RLY07 cells transformed with the different TRP1-M constructs and a plasmid encoding the CMV protease or the empty vector were inoculated in 3 ml of 2% galactose -ura -leu medium and grown at 30° C. to OD₆₀₀ 1. They were washed with 5 ml H₂O and resuspended in 2% galactose -ura -leu -trp medium supplemented with 10% glycerol (=growth selection medium, glycerol promotes CMV protease dimerisation and subsequent proteolytic activity) and diluted to a start OD₆₀₀ 0.01. For the experiments with the coxackievirus 3C protease, preculture medium was 2% glucose -ura -leu, assay medium was 2% glucose -ura -leu trp, and inoculation OD₆₀₀ 0.001. At time zero, assay cultures at the aforesaid start OD₆₀₀ were distributed in 96-well microtiter plates, with a volume of 150 μl per well, and incubated without shaking at 30° C. At the time points indicated in the “results” section, plates were shaken to resuspend cells before being submitted to light scattering measurement at 595 nm in a Tecan Genios reader for determining cell density.

CMV protease inhibitors BI31 and BI36 (Boeringher Ingelheim, Quebec) were dissolved in DMSO and added to the assay cultures at time zero of the growth assay. Final DMSO concentration was 1%.

1.6. Western Blot Analysis

Yeast whole cell extracts were prepared as described by Burke et al. (Burke, Dawson et al. 2000). Proteins were separated by SDS-PAGE and Western blot analysis was performed according to standard procedures (Ausubel et al., 2003, Current Protocols in Molecular Biology). An HA-monoclonal antibody from Sigma (clone 3F10) was used at a concentration of 30 ng/ml to detect expression of TRP1(194)-M.

2. Results

2.1. Insertion of the CMV Protease Cleavage Sequence at 5 Different Locations in Trp1p

Three conditions are critical for appropriate functioning of the above described system: i) The inserted cleavage sequence does not affect enzymatic properties of the Trp1 protein. ii) The cleavage sequence is cleaved by the protease. iii) Cleavage must result in functional inactivation of the Trp enzyme. Indeed, cleavage might occur without separating the two fragments generated and then without impairing the enzymatic function.

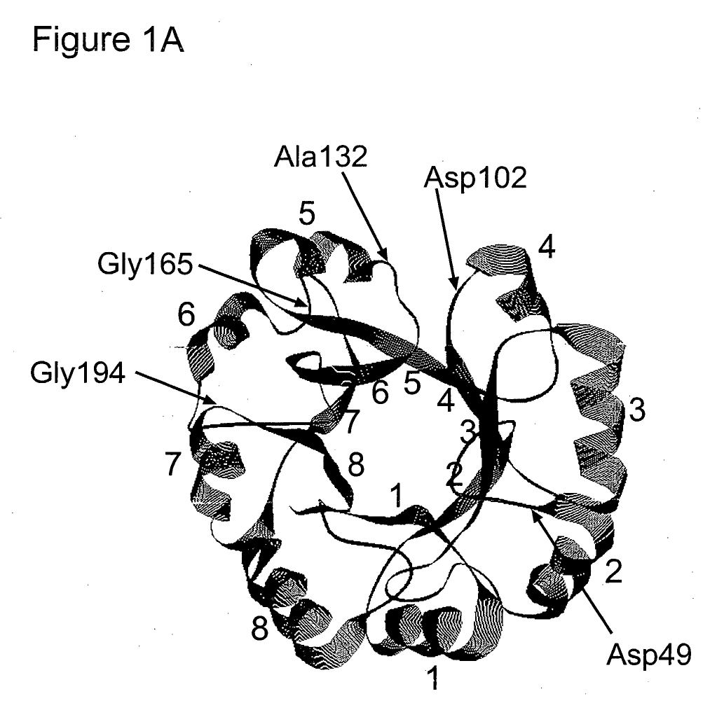

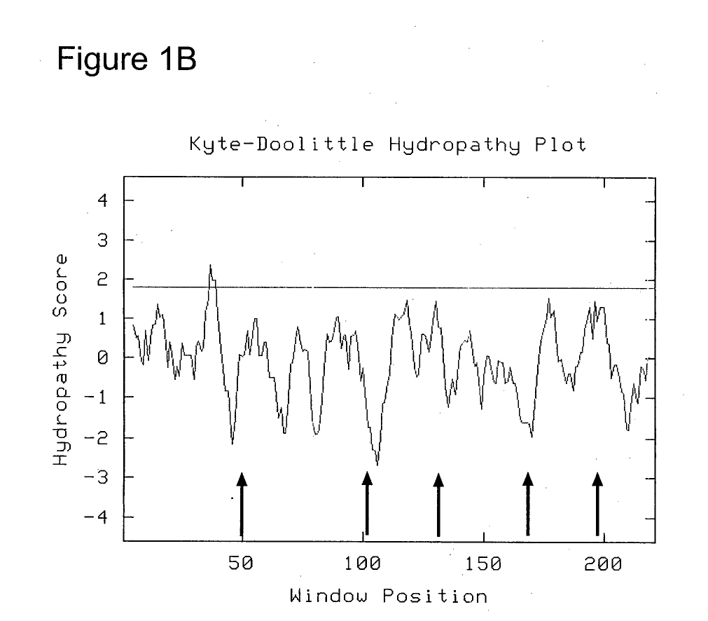

The Trp1 enzyme is a member of the prominent class of proteins that fold into a (β/α)₈-barrel, which is the most commonly occurring fold among enzymes. The core of β/α barrel proteins consists of an eight-stranded parallel β-barrel held together by an extensive β-sheet hydrogen-bonding network. The individual β-strands are usually followed by α-helices that form an outer ring surrounding the cylindrical surface of the central β-barrel (Eder and Wilmanns 1992) (FIG. 1A). The S. cerevisiae Trp1p structure has not yet been determined, but amino acid sequence alignments with the N-(5′-phosphoribosyl)-anthranilate isomerase from E. coli (ePRAI) and Thermotoga maritima (tPRAI) provide us with a reliable model. S. cerevisiae Trp1p shares 28% identical amino acids with E. coli and 33% with T. maritima Trp1p. Alignment and modeling for S. cerevisiae Trp1p was performed with the SWISS-MODEL protein modelling server (Guex and Peitsch 1997; Schwede, Kopp et al. 2003).

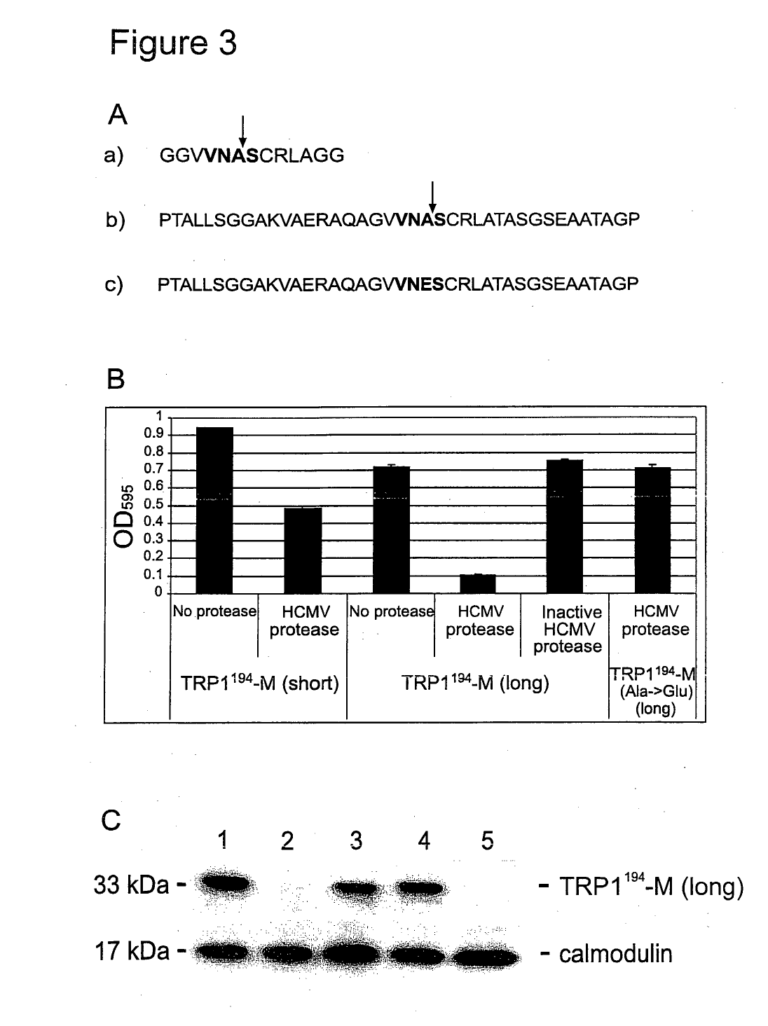

To determine suitable sites for insertion of the cleavage sequence, several constructs were designed. Since turn sequences are in general highly mutable in (β/α)₈-barrels, 5 insertion sites were chosen that are located in such turns between an α-helix and a β-sheet, after amino acids Asp(49), Asp(102), Ala(132), Gly(165) and Gly(194) (FIG. 1). Respective constructs will therefore be referred to as Trp1(49)-M, Trp1(102)-M, Trp1(132)-M, Trp1(165)-M, and Trp1(194)-M. In addition, 4 out of the 5 sites are, according to Kyte-Doolittle, situated in hydrophilic regions, increasing the probability of being located at the periphery of the protein, thereby increasing the probability of the protease to access those sites. The inserted sequence consists of 13 amino acids derived from the M-site (FIG. 3A, a). This site has previously been used in a viral protease assay based on Gal4p inactivation in mammalian cells (Lawler and Snyder 1999). In that assay, increasing amounts of expressed CMV protease caused a gradual reduction of reporter gene expression.

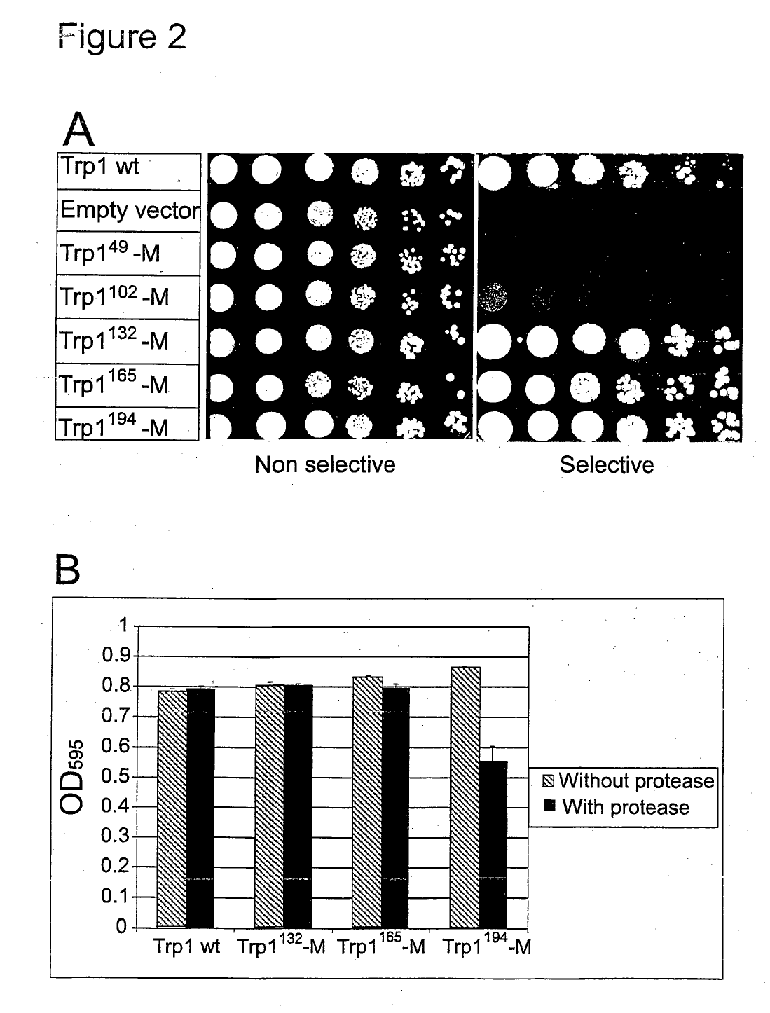

In order to evaluate functionality of the Trp1(49)-M, Trp1(102)-M, Trp1(132)-M, Trp1(165)-M and Trp1(194)-M fusion proteins, a spotting assay was performed. Tryptophan auxotrophic RLY07 cells were trans-formed with wild-type Trp1p (positive control), empty vector (negative control), Trp(49)-M, Trp1(102)-M, Trp1(132)-M, Trp1(165)-M and Trp1(194)-M, and serial dilutions were spotted on selective medium lacking tryptophan and incubated for 3 days at 30° C. For Trp1(132)-M, Trp1(165)-M and Trp1(194)-M expressing cells, growth was indistinguishable from cells expressing wild-type Trp1p, indicating that M-site insertion did not interfere with functionality of the enzyme (FIG. 2A, lanes 1,5,6,7). This is opposed to Trp1(49)-M and Trp1(102)-M constructs that produced non-functional enzymes, as demonstrated by likewise transformed cells unable to grow on selective plate (FIG. 2A, lanes 3,4).

2.2. Site-Specific Cleavage of Trp1194-M by the CMV Protease

Next it was investigated whether the 3 functional Trp1-M proteins were cleaved and inactivated by the CMV protease: Trp1(132)-M, Trp1(165)-M and Trp1(194)M were co-expressed with the CMV protease in the RLY07 strain and cell proliferation was assayed by measuring OD₆₀₀ of the respective transformed cells cultured in liquid selective medium. After 36 hours, Trp1(194)M expressing cells exhibited an OD₆₀₀ reduction of 35% compared to control cells that contained an empty plasmid instead of the protease-expressing plasmid (FIG. 2B, lane 4). We conclude that cleavage of the Trp1(194)-M substrate between helix α7 and strand β8 reduces activity of the Trp1 enzyme. Importantly, this region is situated between two neighbouring loops (loops between β7/α7 and β8/α8) that have been shown to be important for binding of the substrate phosphate ion (Wilmanns, Hyde et al. 1991). A structure disruption in this region is most likely detrimental to phosphate binding of the anthranilate substrate. As opposed to Trp1(194)-M cells, Trp1(132)-M and Trp1(165)-M expressing cells did not show growth reduction despite the fact that CMV protease was expressed and active in those cells (FIG. 2B, lanes 2,3). Thus, the latter 2 engineered Trp1 substrates were either not cleaved or, alternatively, they were cleaved but the separated fragments still form an active enzyme.

To improve cleavage frequency at the M-site of the Trp1(194)-M substrate, the 13 amino acid target sequence was replaced by a longer sequence consisting of 39 amino acids (FIG. 3A, b). Cells expressing the modified Trp1(194)-M together with the active protease showed 85% proliferation reduction (FIG. 3B, lanes 3, 4) when grown in medium lacking tryptophan for 38 h as compared to the 35% proliferation reduction with the original, shorter cleavage site (FIG. 3B, lanes 1, 2). This indicates that the extended recognition site is more efficiently cleaved by the CMV protease.

The CMV protease has been published to hydrolyse both the M-site and R-site between an alanine and a serine (Burck, Berg et al. 1994). To demonstrate site-specific cleavage of the Trp1(194)-M substrate at the M-site, the following experiment was performed: The alanine of the scissile bond was substituted with a glutamic acid (FIG. 3A, c), a mutation known to prevent cleavage (Welch, McNally et al. 1993). As expected, proliferation of cells co-expressing the mutant Trp1(194)-M (A→E) with the protease was comparable to proliferation of cells expressing Trp1(194)-M alone, indicating that the CMV protease cleaves the Trp1(194)-M substrate in sequence-specific manner at the scissile bond (FIG. 3B, lanes 5). Importantly, an inactive version of the CMV protease, harbouring the S(132) A mutation at the catalytic site (Chen, Tsuge et al. 1996), was not able to cleave the Trp1(194)M substrate (FIG. 3B, lane 6).

To provide biochemical evidence for cleavage of the substrate by the CMV protease, an HA tag was cloned both to the N-terminus and C-terminus of Trp1(194)-M. The Trp1 polypeptides were detected in protein extracts from cells transformed with plasmids encoding different Trp1-Mp substrates by Western blot analysis using an anti-HA antibody. The full-length substrate migrates at 33 kDa (FIG. 3C, lane 1). Co-expression of active CMV protease (lane 2) causes disappearance of the full-length substrate. However, no cleaved fragments could be detected, probably due to either degradation or too low detection threshold. Indeed, since the Trp1(194)-M construct is expressed from a weak promoter (a 5′ truncated version of the ADH promoter), the intracellular concentration of the fragments is most likely very low. Lane 3 provides biochemical evidence that the inactive CMV protease does not cleave the Trp1(194)-M substrate (since the full-length substrate band does not disappear), and lane 4, that active protease has no effect on the point-mutated Trp1(194)-M (A→E) substrate, as the band is also present. The use of the calmodulin antibody serves as an internal control for protein amounts.

Taken together, the above experiments show that the Trp1(194)-M substrate is cleaved in a sequence-specific manner by the CMV protease and that this cleavage results in a slow-growth phenotype.

2.3. A Gradual Increase of CMV Protease Expression Level Results in a Gradual Reduction of Cell Growth

The yeast-based system described in this report was developed to identify inhibitors of CMV protease activity in HTS format. To validate sensitivity of the system to different intracellular CMV protease activity levels, the protease was cloned behind series of GALL promoters. Whereas CMV protease in the above experiments was expressed from the full-length (100%) GAL1 promoter, we subcloned the protease on truncated GAL1 promoters, reaching 71%, 46% and 16% of the protein production as compared to the full length GAL1 promoter. This plasmid series was co-expressed with the Trp1(194)-M substrate, and cell growth was measured after 36 hrs at OD₆₀₀. As shown in FIG. 4, a gradual increase of promoter strength, and thus of intracellular protease activity, is inversely proportional to cell proliferation. For example, a reduction of 29% of protease expression (from the 100% promoter to the 71% promoter) results in a 53% stimulation of cell proliferation. A reduction of 54% of protease expression caused a likewise stimulation of 138%. Therefore, even weak inhibitors causing only a partial reduction of CMV protease activity should be detectable in the system.

2.4. Validated CMV Protease Inhibitors Specifically Stimulate Cell Growth in a HTS Format

To further validate the Trp1(194)-M system we challenged it with 2 known human CMV protease inhibitors. Since yeast cells have evolved efficient mechanisms to pump out small chemical compounds, the three major ATP-binding cassette (ABC) transporters Snq2p, Pdr5p and Yor1p (Rogers, Decottignies et al. 2001) were deleted in the strain JPY5 to generate RLY07. It has been shown that deletion of these so-called drug efflux pumps increases sensitivity of yeast cells towards small molecules, allowing to perform screenings in yeast at lower concentrations.

CMV protease inhibitors BI31 (I) and BI36 (II) from Boehringer Ingelheim were applied to our selection system (Yoakim, Ogilvie et al. 1998). Both compounds are built on a β-lactam scaffold.

Lactam derivatives have initially been published as inhibitors of classical serine proteases, such as human leukocyte elastase. Development of such scaffolds by rational design then delivered specific inhibitors of the CMV protease (Finke, Shah et al. 1995). Both compounds show IC₅₀ values of ˜1 μM in an enzymatic assay and inhibit viral replication in cell culture with EC50 values of ˜80 μM (Yoakim, Ogilvie et al. 1998).

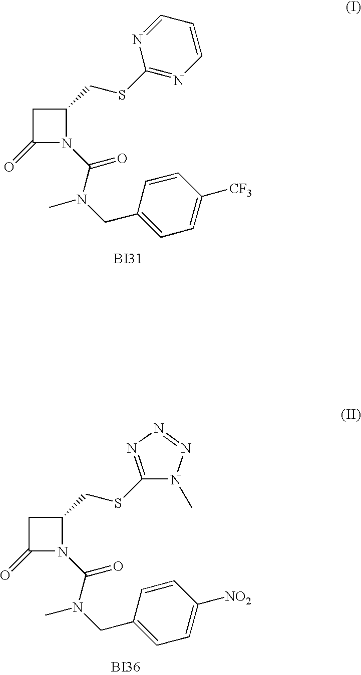

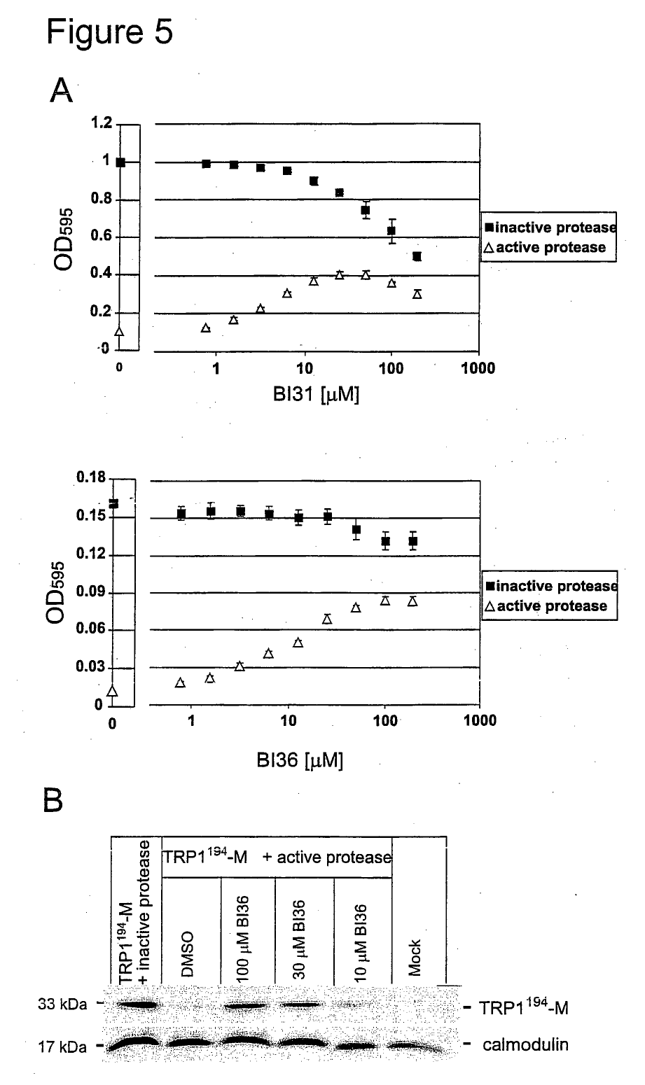

RLY07 cells co-expressing Trp1¹⁹⁴-M substrate and the CMV protease were incubated with a concentration series of BI31 and BI36 in 96-well microtiter plates and cultivated under selective conditions. After ˜2 days incubation at 30° C., increasing concentrations of both BI31 and BI36 caused a dose-dependent increase of cell proliferation (FIG. 5A, triangles). For BI36 an EC₅₀ of 31 μM in the yeast assay was calculated, suggesting that the sensitivity of this assay is similar to the antiviral assay in cell culture (Yoakim, Ogilvie et al. 1998). At a concentration of 100 μM BI36, OD₆₀₀ was close to OD₆₀₀ of cells expressing the inactive protease (squares), meaning that the CMV protease was almost completely inhibited. It should be noted that increasing concentrations of BI31 in RLY07 cells expressing the inactive, point-mutated CMV, protease (squares) causes a gradual decrease of cell proliferation, indicating that BI31 exerts a dose-dependent toxic effect on the cells. Importantly, despite this toxicity BI31 still stimulates growth of cells expressing the active protease (triangles). For example, at 50 μM cell density is multiplied by a factor 4 despite 25% toxicity. This suggests that in a HTS screening compounds will be scored as positives even if they exert some intrinsic toxicity.

The Western blot was performed to provide biochemical evidence for inhibition of cleavage of CMV protease by compound BI36 (FIG. 5B). A 33 kDa band corresponds to the full-length Trp1(194)-M substrate upon co-expression with inactive CMV protease (lane 1). However, co-expressing the active protease instead of the inactive version causes disappearance of the 33 kDa band (lane 2), due to cleavage at the M-site. As in the Western blot on FIG. 3C, unfortunately no cleavage products could be detected.

Application of a concentration series of BI36, 100 μM (lane 3), 30 μM (lane 4), and 10 μM (lane 5), prevented substrate cleavage in a dose-dependent manner. Whereas with 10 μM of BI36 proteolysis is only slightly inhibited, treatment with 100 μM of BI36 inhibits the cleavage almost completely, which is consistent with the determined EC₅₀ of 31 μM in the yeast-based assay.

2.5. The Trp1-M System can be Applied for Other Intracellular Proteases

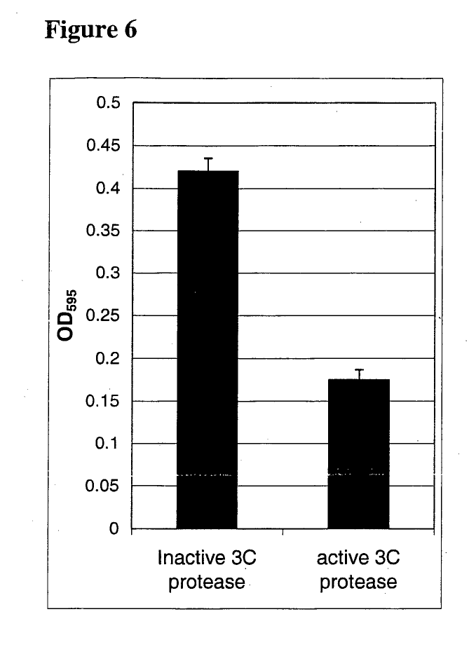

In order to test whether the above described system can be adapted for other proteases, the 39 amino acid M-site in the Trp1(94)M substrate was substituted with a 13 amino acid sequence derived from the 2C/3A cleavage site of the cysteine protease 3C from coxsackievirus B3, resulting in the Trp1-2C/3A substrate. Coxsackievirus (CV) is an enterovirus from the Picornaviridae family. Its RNA genome encodes a single polyprotein of roughly 2200 amino acids that is processed by the viral proteases 2A and 3C. Protease 3C, responsible for the majority of the cleavage events, plays a major role during the virus replication cycle. The Trp1-2C/3A substrate was co-expressed with the 3C protease in RLY07 cells, and cell proliferation was assessed in selective medium lacking tryptophan after 27 h at 30° C. Co-expression of active 3C protease reduced cell growth by 60% compared to cells expressing only the Trp1-2C/3A substrate (FIG. 6), suggesting cleavage of the substrate by the protease. This experiment shows that the Trp1 selection concept can be applied to further proteases apart from the CMV protease.

While there are shown and described presently preferred embodiments of the invention, it is to be distinctly understood that the invention is not limited thereto but may be otherwise variously embodied and practiced within the scope of the following claims.

### REFERENCES

Barberis, A. (2002). “Cell-based high-throughput screens for drug discovery.” European BioPharmaceutical Review (winter).
Batra, R. (2001). “Molecular mechanism for dimerization to regulate the catalytic activity of human cytomegalovirus protease. [see comments.].” Nat Struct Biol 8(9)(September): 810-7.
Baum, E. Z., G. A. Bebernitz, et al. (1993). “Expression and analysis of the human cytomegalovirus UL80-encoded protease: identification of autoproteolytic sites.” Journal of Virology 67(1): 497-506.
Belkhiri, A., V. Lytvyn, et al. (2002). “A noninvasive cell-based assay for monitoring proteolytic activity within a specific subcellular compartment.” Anal Biochem 306(2): 237-46.
Bonneau, P. R., C. Grand-Maitre, et al. (1997). “Evidence of a conformational change in the human cytomegalovirus protease upon binding of peptidylactivated carbonyl inhibitors.” Biochemistry 36(41): 12644-52.
Botstein, D., S. A. Chervitz, et al. (1997). “Yeast as a model organism.” Science 277(5330): 1259-60.
Brenner, C. (2000). “A cultivated taste for yeast.” Genome Biol 1(1): REVIEWS103. Epub 2000 Apr. 27.
Burck, P. J., D. H. Berg, et al. (1994). “Human cytomegalovirus maturational proteinase: expression in Escherichia coli, purification, and enzymatic characterization by using peptide substrate mimics of natural cleavage sites.” Journal of Virology 68(5): 2937-46.
Burke, D., D. Dawson, et al. (2000). Methods in Yeast Genetics. Cole Spring Harbor, N.Y.
Chen, P., H. Tsuge, et al. (1996). “Structure of the human cytomegalovirus protease catalytic domain reveals a novel serine protease fold and catalytic triad.” Cell 86(5): 835-43.
Chrusciel, R. A. and J. W. Strohbach (2004). “Non-peptidic HIV protease inhibitors.” Curr Top Med Chem 4(10): 1097-114.
Dasmahapatra, B., B. DiDomenico, et al. (1992). “A genetic system for studying the activity of a proteolytic enzyme.” Proc Natl Acad Sci USA 89(9): 4159-62.
Dasmahapatra, B., E. J. Rozhon, et al. (1991). “Cell-free expression of the coxsackievirus 3C protease using the translational initiation signal of an insect virus RNA and its characterization.” Virus Res 20(3): 237-49.
Eder, J. and K. Kirschner (1992). “Stable substructures of eightfold beta alpha-barrel proteins: fragment complementation of phosphoribosylanthranilate isomerase.” Biochemistry 31(14): 3617-25.
Eder, J. and M. Wilmanns (1992). “Protein engineering of a disulfide bond in a beta/alpha-barrel protein.” Biochemistry 31(18): 4437-44.
Fehrenbacher, N. and M. Jaattela (2005). “Lysosomes as targets for cancer therapy.” Cancer Res 65(8): 2993-5.
Fields, S, and O, Song (1989). “A novel genetic system to detect protein-protein interactions.” Nature 340(6230): 245-6.
Finke, P. E., S. K. Shah, et al. (1995). “Orally active beta-lactam inhibitors of human leukocyte elastase. 3. Stereospecific synthesis and structure-activity relationships for 3,3-dialkylazetidin-2-ones.” J. Med. Chem. 38(13): 2449-62.
Fried, H. M., and J. R. Warner (1982). “Molecular cloning and analysis of yeast gene for cycloheximide resistance and ribosomal protein L29”. Nucleic Acid. Res. 10: 3133-3148.
Gibson, W. (2001). “Action at the assemblin dimer interface. [letter; comment.].” Nat Struct Biol 8(9): 739-741.
Gietz, D., A. St Jean, et al. (1992). “Improved method for high efficiency transformation of intact yeast cells.” Nucleic Acids Res 20(6): 1425.
Gietz, R. D. and A. Sugino (1988). “New yeast-Escherichia coli shuttle vectors constructed with in vitro-mutagenized yeast genes lacking six-base pair restriction sites.” Gene 74(2): 527-34.
Guex, N. and M. C. Peitsch (1997). “SWISS-MODEL and the Swiss-PdbViewer: an environment for comparative protein modeling.” Electrophoresis 18(15): 2714-23.
Gunde, T., S. Tanner, et al. (2004). “Quenching accumulation of toxic galactose-1-phosphate as a system to select disruption of protein-protein interactions in vivo”. BioTechniques 37(5): 844-51.
Hennig, M., R. Sterner, et al. (1997). “Crystal structure at 2.0 A resolution of phosphoribosyl anthranilate isomerase from the hyperthermophile Thermotoga maritima: possible determinants of protein stability.” Biochemistry 36(20): 6009-16.
Hilleman, D. E. (2000). “Role of angiotensin-converting-enzyme inhibitors in the treatment of hypertension.” Am J Health Syst Pharm 57(Suppl 1): S8-11.
Holwerda, B. C. (1997). “Herpesvirus proteases: targets for novel antiviral drugs.” Antiviral Research 35(1): 1-21.
Hommel, U., M. Eberhard, et al. (1995). “Phosphoribosyl anthranilate isomerase catalyzes a reversible amadori reaction.” Biochemistry 34(16): 5429-39.
Hoog, S. S., W. W. Smith, et al. (1997). “Active site cavity of herpesvirus proteases revealed by the crystal structure of herpes simplex virus protease/inhibitor complex.” Biochemistry 36(46): 14023-9.
Hughes, T. R. (2002). “Yeast and drug discovery.” Funct Integr Genomics 2(4-5): 199-211. Epub 2002 May 31.
Johnston, P. A. (2002). “Cellular platforms for HTS: three case studies.” Drug Discov Today 7(6): 353-63.
Kemnitzer, W., J. Drewe, et al. (2004). “Discovery of 4-aryl-4H-chromenes as a new series of apoptosis inducers using a cell- and caspase-based high-throughput screening assay. 1. Structure-activity relationships of the 4-aryl group.” J Med Chem 47(25): 6299-310.
Khayat, R., R. Batra, et al. (2003). “Structural and biochemical studies of inhibitor binding to human cytomegalovirus protease.” Biochemistry 42(4): 885-91.
Lawler, J. F., Jr. and S. H. Snyder (1999). “Viral protease assay based on GAL4 inactivation is applicable to high-throughput screening in mammalian cells.” Anal Biochem 269(1): 133-8.
Lee, J. C., Y. F. Shih, et al. (2003). “Development of a cell-based assay for monitoring specific hepatitis C virus NS3/4A protease activity in mammalian cells.” Anal Biochem 316(2): 162-70.
Lindsten, K., T. Uhlikova, et al. (2001). “Cell-based fluorescence assay for human immunodeficiency virus type 1 protease activity.” Antimicrob Agents Chemother 45(9): 2616-22.
Lüthi, U. (2002). “Proteolytic enzymes as therapeutic targets.” European BioPharmaceutical Review (Summer)
Mao, H. X., S. Y. Lan, et al. (2003). “Establishment of a cell-based assay system for hepatitis C virus serine protease and its primary applications.” World Gastroenterol 9(11): 2474-9.
Margosiak, S. A., D. L. Vanderpool, et al. (1996). “Dimerization of the human cytomegalovirus protease: kinetic and biochemical characterization of the catalytic homodimer.” Biochemistry 35(16): 5300-7.
McIntosh, C. H., H. U. Demuth, et al. (2005). “Dipeptidyl peptidase IV inhibitors: how do they work as new antidiabetic agents?” Regul Pept 128(2): 159-65.
Munder, T. and A. Hinnen (1999). “Yeast cells as tools for target-oriented screening.” Appl Microbiol Biotechnol 52(3): 311-20.
Oh, M., S. Y. Kim, et al. (2003). “Cell-based assay for beta-secretase activity.” Anal Biochem 323(1): 7-11.
Pinko, C., S. A. Margosiak, et al. (1995). “Single-chain recombinant human cytomegalovirus protease. Activity against its natural protein substrate and fluorogenic peptide substrates.” J Biol Chem 270(40): 23634-40.
Randolph, J. T. and D. A. DeGoey (2004). “Peptidomimetic inhibitors of HIV protease.” Curr Top Med Chem 4(10): 1079-95.
Rogers, B., A. Decottignies, et al. (2001). “The pleitropic drug ABC transporters from Saccharomyces cerevisiae.” J Mol Microbiol Biotechnol 3(2): 207-14.
Schwede, T., J. Kopp, et al. (2003). “SWISS-MODEL: An automated protein homology-modeling server.” Nucleic Acids Res 31(13): 3381-5.
Sheaffer, A. K., W. W. Newcomb, et al. (2000). “Evidence for controlled incorporation of herpes simplex virus type 1 UL26 protease into capsids.” J Virol 74(15): 6838-48.
Shieh, H. S., R. G. Kurumbail, et al. (1996).

“Three-dimensional structure of human cytomegalovirus protease. [erratum appears in Nature 1996 Nov. 21; 384(6606):288].” Nature 383(6597): 279-82.
Smith, T. A. and Kohorn, B. D., 1991. “Direct selection for sequences encoding proteases of known specificity”. Proc Natl Acad Sci USA 88: 5159-5162.
Stalker, T. J., Y. Gong, et al. (2005). “The calcium-dependent protease calpain causes endothelial dysfunction in type 2 diabetes.” Diabetes 54(4): 1132-40.
Tong, L. (2002). “Viral proteases.” Chem Rev 102(12): 4609-26.
Toyn, J. L., P. L. Gunyuzlu, et al. (2000). “A counterselection for the tryptophan pathway in yeast: 5-fluoroanthranilic acid resistance”. Yeast 16: 553-560.
Trang, P., K. Kim, et al. (2003). “Expression of an RNase P ribozyme against the mRNA encoding human cytomegalovirus protease inhibits viral capsid protein processing and growth.” J Mol Biol 328(5): 1123-35.
Waxman, L. and P. L. Darke (2000). “The herpesvirus proteases as targets for antiviral chemotherapy.” Antiviral Chemistry & Chemotherapy 11(1): 1-22.
Welch, A. R., L. M. McNally, et al. (1993). “Herpesvirus proteinase: site-directed mutagenesis used to study maturational, release, and inactivation cleavage sites of precursor and to identify a possible catalytic site serine and histidine.” J Virol 67(12): 7360-72.
Welch, A. R., A. S. Woods, et al. (1991). “A herpesvirus maturational proteinase, assemblin: Identification of its gene, putative active site domain, and cleavage site.” Proc Natl Acad Sci USA 88: 10792-10796.
Wilmanns, M., C. C. Hyde, et al. (1991). “Structural conservation in parallel beta/alpha-barrel enzymes that catalyze three sequential reactions in the pathway of tryptophan biosynthesis.” Biochemistry 30(38): 9161-9.
Wittwer, A. J., C. L. Funckes-Shippy, et al. (2002). “Recombinant full-length human cytomegalovirus protease has lower activity than recombinant processed protease domain in purified enzyme and cell-based assays.” Antiviral Research 55(2): 291-306.
Yoakim, C., W. W. Ogilvie, et al. (1998). “Potent beta-lactam inhibitors of human cytomegalovirus protease.” Antivir Chem Chemother 9(5): 379-87.
Zuck, P., E. M. Murray, et al. (2004). “A cell-based beta-lactamase reporter gene assay for the identification of inhibitors of hepatitis C virus replication.” Anal Biochem 334(2): 344-55.

1. A non-regulatory tester polypeptide for monitoring protease activity, which—comprises the sequence of a marker protein whose activity can be detected by positive and/or negative growth selection and an additional sequence, said additional sequence being inserted at a specific permissible site in a surface loop of said marker protein and comprising a cognate cleavage sequence for a protease, and
is inactivated upon cleavage by said protease.

2. The polypeptide of claim 1 wherein the marker protein is a cytoplasmic protein.

3. The polypeptide of claim 1 wherein the marker protein is a biosynthetic enzyme for an essential cellular compound.

4. The polypeptide of claim 1 with the marker protein being an auxotrophy marker for both positive and negative selection.

5. The polypeptide of claim 1 wherein the marker protein is an enzyme of an amino acid biosynthesis pathway.

6. The polypeptide of claim 1 wherein the marker protein is the yeast Trp1p protein.

7. The polypeptide of claim 6 comprising a protease cleavage sequence inserted after Gly194 of Trp1p.

8. The polypeptide of claim 1, characterized in that the cleavage sequence is between about 5-39 amino acids long.

9. The polypeptide of claim 1, characterized in that the protease cleavage sequence is selected from the group consisting of SEQ. ID. NO: 2=GGVVNASCRLAGG,
SEQ. ID. NO: 3=KVAERANAGWQASCRLATAS and
SEQ. ID. NO: 4=PTALLSGGAKVAERAQAGVVNASCRLATASGSEAATAGP.

10. The polypeptide of claim 1, characterized in that it is susceptible to cleavage by a viral protease.

11. The polypeptide of claim 10 that is susceptible to CMV protease.

12. The polypeptide of claim 1 characterized in that the additional sequence comprising the cleavage sequence is the sequence of an autoprotease.

13. The polypeptide of claim 1 that is susceptible to coxsackievirus protease 3C.

14. The polypeptide of claim 1 that is modified by one or more point mutations.

15. The polypeptide of claim 8 wherein the point mutations are within the natural, cognate cleavage sequence of a protease.

16. A nucleic acid encoding the polypeptide of claim 1.

17. A nucleic acid according to claim 16 comprising a promoter for expression of the tester polypeptide.

18. A recombinant vector comprising the nucleic acid of claim 16.

19. A prokaryotic or eukaryotic cell comprising the nucleic acid of claim 16 and a protease capable of cleaving the tester polypeptide within the cognate cleavage sequence for said protease.

20. (canceled)

21. The cell according to claim 19, which is a yeast cell.

22. A method to identify a protease inhibitor comprising the steps of
providing a cell according to claim 19,
exposing said cell to candidate inhibitor substances,
growing said cell under conditions that are non-permissive for cell proliferation in the presence of a functional protease, but permissive for cell proliferation in the additional presence of an inhibitor of said protease, and
selecting an inhibitor on the basis of cell proliferation.

23. A method to identify a suitable site in a non-regulatory marker protein for insertion of a protease cleavage sequence, said marker protein being suitable for positive as well as negative selection, said method comprising the steps of
identifying putative surface loops in said marker protein,
providing an expression vector comprising a nucleic acid encoding said marker protein,
inserting a nucleic acid comprising a coding sequence for said protease cleavage sequence at a random position within the coding sequence of said putative surface loops, resulting in a plasmid comprising a gene encoding a tester protein according to claim 1,
transforming with said plasmid a yeast cell comprising a protease that is capable of cleaving said protease cleavage sequence,
growing transformants in the presence of a specific inhibitor of said protease under conditions requiring a function of said tester protein,
shifting growing clones to conditions non-permissive for a function of said tester protein and lacking said inhibitor,
determining the nucleic acid sequence of the gene encoding said tester protein of a surviving clone.

24. A method to identify a suitable site in a non-regulatory marker protein for insertion of a protease cleavage sequence, said marker protein being suitable for positive as well as negative selection, said method comprising the steps of
identifying putative surface loops in said marker protein,
providing an expression vector comprising a nucleic acid encoding said marker protein,
inserting a nucleic acid comprising a coding sequence for said protease cleavage sequence at a random position within the coding sequence of anyone of said putative surface loops, resulting in a plasmid comprising a gene encoding a tester protein according to claim 1,
transforming with said plasmid a yeast cell comprising a gene encoding a protease that is capable of cleaving said protease cleavage sequence, said gene being under the control of a tightly regulated promoter,
growing transformants under repressing or non-inducing conditions with respect to said promoter and under conditions requiring a function of said tester protein,
shifting growing cells to derepressing or inducing conditions with respect to said promoter for protease expression and non-permissive conditions with respect to a function of said tester protein,
determining the nucleic acid sequence of the gene encoding said tester protein of a growing cell.

25. A method to identify a suitable site in a non-regulatory marker protein for insertion of a protease cleavage sequence, said marker protein being suitable for positive as well as negative selection, said method comprising the steps of
identifying putative surface loops in said marker protein,
providing an expression vector comprising a nucleic acid encoding said marker protein,
inserting a nucleic acid comprising a coding sequence for said protease cleavage sequence at a random position within the coding sequence of anyone of said putative surface loops, resulting in a plasmid comprising a gene encoding a tester protein according to claim 1,
providing a first yeast cell comprising a protease capable of cleaving said cleavage sequence and a second yeast cell lacking said protease,
transforming said first yeast cell with said plasmid and growing transformants under non-permissive conditions with respect to a function of said tester protein,
isolating said plasmid from a surviving cell,
transforming said second yeast cell with said isolated plasmid and growing transformants under conditions requiring a function of said tester protein,
determining the nucleic acid sequence of said gene encoding said tester protein of a growing cell.

26. A method to identify a suitable site in a non-regulatory marker protein for insertion of a protease cleavage sequence, said marker protein being suitable for positive as well as negative selection, said method comprising the steps of
identifying putative surface loops in said marker protein,
providing an expression vector comprising a nucleic acid encoding said marker protein,
inserting a nucleic acid comprising a coding sequence for said protease cleavage sequence at a random position within the coding sequence of anyone of said putative surface loops, resulting in a plasmid comprising a gene encoding a tester protein according to claim 1,
providing a first yeast cell comprising a protease capable of cleaving said cleavage sequence and a second yeast cell lacking said protease,
transforming said second yeast cell with said plasmid and growing transformants under conditions requiring a function of said tester protein,
isolating said plasmid from a growing cell,
transforming said first cell with said isolated plasmid and growing transformants under conditions non-permissive for a function of said tester protein,
determining the nucleic acid sequence of said gene encoding said tester protein of a surviving cell.

27. A method to identify a suitable site in a non-regulatory marker protein for insertion of a protease cleavage sequence, said marker protein being suitable for positive as well as negative selection, said method comprising the steps of
identifying putative surface loops in said marker protein,
providing an expression vector comprising a nucleic acid encoding said marker protein,
inserting a nucleic acid comprising a coding sequence for said protease cleavage sequence at a random position within the coding sequence of anyone of said putative surface loops, resulting in a plasmid comprising a gene encoding a tester protein according to claim 1,
providing a yeast cell lacking a protease capable of cleaving said cleavage sequence,
transforming said yeast cell with said plasmid and selecting for growth under conditions requiring a function of said tester protein, obtaining transformants,
providing a second plasmid capable of expressing a gene encoding said protease,
transforming said transformants with said second plasmid and selecting for growth under conditions non-permissive for a function of said tester protein,
determining the nucleic acid sequence of said gene encoding said tester protein of a surviving cell.

28. A method to identify the cleavage site of ease comprising the steps of
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid encoding a tester protein according to claim 1,
transforming with said plasmid a suitable host cell comprising said protease
growing transformants in the presence of a specific inhibitor of said protease under conditions requiring a function of said tester protein,
shifting growing clones to conditions non-permissive for a function of said tester protein and lacking said inhibitor,
determining the nucleic acid sequence of the gene encoding said tester protein of a surviving clone.

29. A method to identify the cleavage site of a known protease comprising the steps of
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid comprising a gene encoding a tester protein according to claim 1,
transforming with said plasmid a suitable host cell comprising the gene encoding said protease under a control of a tightly regulated promoter,
growing transformants under repressing or non-inducing conditions with respect to said promoter and under conditions requiring a function of said tester protein,
shifting growing cells to derepressing or inducing conditions with respect to said promoter and non-permissive conditions with respect to a function of said tester protein,
determining the nucleic acid sequence of the gene encoding said tester protein of a surviving cell.

30. A method to identify the cleavage site of a known protease comprising the steps of
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid comprising a gene encoding a tester protein according to claim 1,
providing a first yeast cell comprising a protease capable of cleaving said cleavage sequence and a second yeast cell lacking said protease,
transforming said first yeast cell with said plasmid and growing transformants under non-permissive conditions with respect to a function of said tester protein,
isolating said plasmid from a surviving cell,
transforming said second cell with said isolated plasmid and growing transformants under conditions requiring a function of said tester protein,
determining the nucleic acid sequence of the gene encoding said tester protein of a growing cell.

31. A method to identify the cleavage site of a known protease comprising the steps of
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid comprising a gene encoding a tester protein according to claim 1,
providing a first yeast cell comprising a protease capable of cleaving said cleavage sequence and a second yeast cell lacking said protease,
transforming said second yeast cell with—said plasmid and growing transformants under conditions requiring a function of said tester protein,
isolating said plasmid from a growing cell,
transforming said first yeast cell with said isolated plasmid and growing transformants under non-permissive conditions with respect to a function of said tester protein,
determining the nucleic acid sequence of said gene encoding said tester protein of a surviving cell.

32. A method to identify the cleavage site of a known protease comprising the steps of
providing an expression vector encoding a non-regulatory marker protein suitable for positive as well as negative selection with at least one known permissible site in a surface loop for the insertion of a sequence,
inserting a coding sequence for about 5-39 amino acids into said site, resulting in a plasmid comprising a gene encoding a tester protein according to claim 1,
providing a yeast cell lacking a protease capable of cleaving said cleavage sequence,
transforming said yeast cell with said plasmid and selecting for growth under conditions requiring a function of said tester protein, obtaining transformants,
providing a second plasmid capable of expressing a gene encoding said protease, transforming said transformants with said second plasmid and selecting for growth under conditions non-permissive with respect to a function of said tester protein,
determining the nucleic acid sequence of said gene encoding said tester protein of a surviving cell.

33. A method to identify a protease showing improved activity and/or changed specificity or a derivative of said protease, comprising the steps of
providing cells expressing a functional, non-regulatory tester polypeptide suitable for negative selection,
providing an expression library comprising putative genes encoding said protease,
transforming said cells with said expression library,
growing transformants under non-permissive conditions with respect to a function of said tester protein,
identifying among surviving clones those which lack full-length tester polypeptide,
determining from identified clones the nucleic acid sequence of the gene encoding said protease.


SEQUENCE LISTING (38 sequences)

SEQ ID NO: 1 - protein, 10 aa, artificial
  protease cleavage sequence
  human CMV protease cleavage sequence (1)..(10)
GVVNASCRLA

SEQ ID NO: 2 - protein, 13 aa, artificial
  protease cleavage sequence
  human CMV protease cleavage sequence (1)..(13)
GGVVNASCRLAGG

SEQ ID NO: 3 - protein, 21 aa, artificial
  protease cleavage sequence
  human CMV protease cleavage sequence (1)..(21)
KVAERANAGVVQASCRLATAS

SEQ ID NO: 4 - protein, 39 aa, artificial
  protease cleavage sequence
  human CMV protease cleavage sequence (1)..(39)
PTALLSGGAKVAERAQAGVVNASCRLATASGSEAATAGP

SEQ ID NO: 5 - protein, 4 aa, artificial
  protease cleavage sequence
  human CMV protease cleavage sequence (1)..(4): X = any amino acid
VXAS

SEQ ID NO: 6 - protein, 4 aa, artificial
  protease cleavage sequence
  human CMV protease cleavage sequence (1)..(4): X = any amino acid
LXAS

SEQ ID NO: 7 - protein, 4 aa, artificial
  protease cleavage sequence
  human CMV protease cleavage sequence (1)..(4): X = any amino acid
IXAS

SEQ ID NO: 8 - protein, 11 aa, artificial
  protease cleavage sequence
  HSV-1 protease cleavage sequence (1)..(11)
ALVNASSAAHV

SEQ ID NO: 9 - protein, 10 aa, artificial
  protease cleavage sequence
  protease cleavage sequenceVZV (1)..(10)
QDVNAVEASS

SEQ ID NO: 10 - protein, 11 aa, artificial
  protease cleavage sequence
  protease cleavage sequenceEBV (1)..(11)
KLVQASASGVA

SEQ ID NO: 11 - protein, 7 aa, artificial
  protease cleavage sequence
  HHV-6 protease cleavage sequence (1)..(7)
PSILNAS

SEQ ID NO: 12 - protein, 8 aa, artificial
  protease cleavage sequence
  HIV-1 protease cleavage sequence (1)..(8)
SFNFPQIT

SEQ ID NO: 13 - protein, 8 aa, artificial
  protease cleavage sequence
  HIV-1 protease cleavage sequence (1)..(8)
TLNFPISP

SEQ ID NO: 14 - protein, 11 aa, artificial
  protease cleavage sequence
  Hepatitis C virus protease (1)..(11)
DLEVVTSTWVL

SEQ ID NO: 15 - protein, 13 aa, artificial
  protease cleavage sequence
  Hepatitits C virus protease cleavage sequence (1)..(13)
  Coxsackievirus 3C protease cleavage sequence (1)..(13)
GTTLEALFQGPPV

SEQ ID NO: 16 - protein, 10 aa, artificial
  protease cleavage sequence
  Rhinovirus C3 protease cleavage sequence (1)..(10)
LEVLFQGPLG

SEQ ID NO: 17 - protein, 8 aa, artificial
  protease cleavage sequence
  SARS coronavirus 3C-like protease (1)..(8)
SAVLQSGF

SEQ ID NO: 18 - protein, 5 aa, artificial
  protease cleavage sequence
  Caspase-1 cleavage sequence (1)..(5)
WFKDS

SEQ ID NO: 19 - protein, 5 aa, artificial
  protease cleavage sequence
  Caspase-1 cleavage sequence (1)..(5)
FEDDA

SEQ ID NO: 20 - protein, 5 aa, artificial
  protease cleavage sequence
  Caspase-1 cleavage sequence (1)..(5)
YVHDA

SEQ ID NO: 21 - protein, 5 aa, artificial
  protease cleavage sequence
  Caspase-1 cleavage sequence (1)..(5)
  Caspase cleavage sequence (1)..(5)
DGPDG

SEQ ID NO: 22 - protein, 5 aa, artificial
  protease cleavage sequence
  Caspase-2 cleavage sequence (1)..(5)
  Caspase cleavage sequence (1)..(5)
DEVDG

SEQ ID NO: 23 - protein, 5 aa, artificial
  protease cleavage sequence
  Caspase-3 cleavage sequence (1)..(5)
IETDS

SEQ ID NO: 24 - protein, 5 aa, artificial
  protease cleavage sequence
  Caspase-3 cleavage sequence (1)..(5)
DEVDN

SEQ ID NO: 25 - protein, 5 aa, artificial
  protease cleavage sequence
  Caspase-3 cleavage sequence (1)..(5)
DMQDN

SEQ ID NO: 26 - protein, 5 aa, artificial
  protease cleavage sequence
  Caspase-3 cleavage sequence (1)..(5)
DEPDS

SEQ ID NO: 27 - protein, 5 aa, artificial
  protease cleavage sequence
  Caspase-3 cleavage sequence (1)..(5)
DEADG

SEQ ID NO: 28 - protein, 5 aa, artificial
  protease cleavage sequence
  Caspase-3 cleavage sequence (1)..(5)
DETDS

SEQ ID NO: 29 - protein, 5 aa, artificial
  protease cleavage sequence
  Caspase-3 cleavage sequence (1)..(5)
DACDT

SEQ ID NO: 30 - protein, 5 aa, artificial
  protease cleavage sequence
VEIDN

SEQ ID NO: 31 - protein, 5 aa, artificial
  protease cleavage sequence
  Caspase-6 cleavage sequence (1)..(5)
  Caspase-8 cleavage sequence (1)..(5)
VETDS

SEQ ID NO: 32 - protein, 5 aa, artificial
  protease cleavage sequence
  Caspase-8 cleavae sequence (1)..(5)
LEMDL

SEQ ID NO: 33 - protein, 8 aa, artificial
  protease cleavage sequence
  Plasmepsin cleavage sequence (1)..(8)
ERMFLSFP

SEQ ID NO: 34 - protein, 6 aa, artificial
  protease cleavage sequence
  Thrombin cleavage sequence (1)..(6)
VPRSFR

SEQ ID NO: 35 - skipped

SEQ ID NO: 36 - protein, 8 aa, artificial
  protease cleavage sequence
  MMP7 cleavage sequence (1)..(8)
GPLGVRGL

SEQ ID NO: 37 - protein, 15 aa, artificial
  protease cleavage sequence
  Bacillus anthracis protease cleavage sequence (1)..(15)
  Bacillus anthracis leathal factore cleavage sequence (1)..(15)
LARRKPVLPALTINP

SEQ ID NO: 38 - protein, 8 aa, artificial
  protease cleavage sequence
  Renin cleavage sequence (1)..(8)
PFHLLVYS